(19) United States
(12) Patent Application Publication
(10) Pub. No.: US 2025/0359555 A1
(43) Pub. Date: Nov. 27, 2025

(54) STREPTOMYCES VENEZUELAE FOR THE BIOLOGICAL CONTROL OF PLANT DISEASES

(71) Applicants: UNIVERSITA' DEGLI STUDI DI MODENA E REGGIO EMILIA (80%), Modena (MO) (IT); CORA SEEDS S.R.L. (20%), Cesena (FC) (IT)

(72) Inventors: Gianmarco Conti Nibali, Modena (MO) (IT); Emilio Stefani, Modena (MO) (IT); Davide Giovanardi, Modena (MO) (IT)

(73) Assignees: UNIVERSITA' DEGLI STUDI DI MODENA E REGGIO EMILIA (80%), Modena (MO) (IT); CORA SEEDS S.R.L. (20%), Cesena (FC) (IT)

(21) Appl. No.: 18/873,372

(22) PCT Filed: Jun. 16, 2023
(86) PCT No.: PCT/IB2023/056252

§ 371 Date: Dec. 10, 2024

(30) Foreign Application Priority Data
Jun. 17, 2022 (IT) 102022000012914

(51) Int. Cl. A01N 63/28; A01N 25/04; A01P 3/00; C12N 1/20; C12R 1/61

(52) U.S. Cl. CPC A01N 63/28; A01N 25/04; A01P 3/00; C12N 1/205; C12R 2001/61

(57) ABSTRACT

The present invention relates to the use of at least one Streptomyces venezuelae strain, in particular the Streptomyces venezuelae DSM 33887 strain, as a phytopharmaceutical drug, in particular as a fungicide or bio-fungicide. A further object of the invention is a Streptomyces venezuelae strain referred to as DSM 33887, and compositions and uses thereof. The present invention relates to a method for the control of phytopathogens and a method to prevent, control, and treat plant diseases caused by phytopathogens, comprising the use of a Streptomyces venezuelae strain, preferably the DSM 33887 strain.

### TECHNICAL FIELD OF THE INVENTION

The present invention relates to the use of at least one Streptomyces venezuelae strain as a phytopharmaceutical drug and methods for the control of phytopathogens to prevent, control, and treat plant diseases caused by them. The invention relates to a Streptomyces venezuelae DSM 33887 strain, compositions and uses thereof.

### BACKGROUND OF THE INVENTION

Agriculture occupies more than ⅓ of the earth's surface (FAOSTAT, 2016). Primary component of the agricultural productive system is the seed, both for perpetrating the genotype, and as an integral component required for food production. For this reason, the seed quality plays an essential role in agriculture: features like health, germination performances, vigor, and purity have to be always guaranteed. The seed treatments and coating are a diffuse practice to guarantee the phytosanitary quality of the seeds and avoid that the seed itself becomes a vehicle of pathogens and, therefore, of diseases.

In the past, a massive use of phytopharmaceutical drugs has been made to guarantee high phytosanitary standards of the seeds, however, the need to guarantee higher sustainability and security to the agricultural systems and the need to have greater respect for the environment are leading to increasingly limiting the use of synthetic phytopharmaceutical drugs that are notoriously harmful to humans and to the environment.

In recent years, the European Commission has progressively banned the use of most active synthetic substances—mostly fungicides—traditionally used for the seed coating. Therefore, to date, phytopharmaceutical drugs efficient for such use no longer exist in the phytosanitary list of authorized plant protection products, excluding the active substance fludioxonil, authorized on cereal seeds, but not authorized on horticultural crops, unless with zonal procedure (i.e., derogation authorization with extension of use to face a phytosanitary emergency).

Therefore, in the last years, the need to find alternative active substances or microorganisms with antibacterial and antifungal activity has increased. In the recent years, some microorganisms were authorized for use as active substances in plant protection. For example, Bacillus thuringiensis for the management of some insect pests; Bacillus amyloliquefaciens for management of several bacterial diseases, grey mold (Botrytis cinerea), and brown rot; Ampelomyces quisqualis for the management of grapevine powdery mildew. None of the registered microorganisms is specifically indicated for seed coating, but all of them should be applied on the foliage of the interested crops.

Considering the above, there is still the need to find alternative microorganisms efficient in the biological control of plant diseases caused by phytopathogens (microorganisms), in particular fungi, preferably suitable for seed coating, and able to contrast infections caused by pathogenic microorganisms, in particular fungi, which live in the soil and are harmful for plants.

### BRIEF DESCRIPTION OF THE FIGURES

FIG. 1 shows the inhibition percentage of fungal growth (IG %) given by the strains: A) Streptomyces sp. DLS1568; B) Streptomyces sp. SA51; C) Pseudomonas sp. PT 65 and D) Agrobacterium sp. AR 39, against phytopathogenic fungi on potato dextrose agar (PDA) medium. Bars indicate the standard deviation of means (±SD) related to the inhibition percentages of fungal growth.

FIG. 2 shows the siderophore production of the Streptomyces sp. DLS1568 and Streptomyces sp. SA51 strains and the positive control (1M pyrocatechol). Means marked with different letters indicate significant difference between the 3 samples according to the statistical analysis (One-way ANOVA, Tukey's Test; p<0.001). Bars indicate the standard deviation value of the means (±SD).

FIG. 3 shows the phosphate solubilization activity for Streptomyces sp. DLS1568 and Streptomyces sp. SA51 and the negative control. Means marked with different letters indicate significant difference between the 3 samples according to the statistical analysis (One-way ANOVA, Tukey's Test, p<0.001). Bars indicate the standard deviation value of the means (±SD).

FIG. 4 shows the germination results expressed as percentage value (%) of the seed samples belonging to: A) onion, B) fennel and treated with Streptomyces sp. DLS1568 and Streptomyces sp. SA51, respectively, and the negative control (uncoated). Means marked by the same letters indicate a not significant difference between two treatment groups according to the statistical analysis (One-way ANOVA, Tukey's test, p>0.05). Means marked by different letters indicate a significant difference according to the statistical analysis result (One-way ANOVA, Tukey's test, p<0.001) between two treatment groups. Bars indicate the standard deviation value of the means (±SD).

FIG. 5 shows the value expressed as disease index, following the experimental inoculum with the phytopathogen fungus F. oxysporum f. sp. raphani (Fus Ruc 9 strain), of the seedlings emerged from seeds coated with Streptomyces sp. DLS1568, Streptomyces sp. SA51, and from uncoated seeds (Control). Data are reported as means ±SD of the disease index. Means marked by the same letters indicate a not significant difference (One-way ANOVA, Tukey's test, p>0.05) between the treatment groups. Means marked by different letters indicate a significant difference (One-way ANOVA, Tukey's test, p<0.001) between the treatment groups. Bars indicate the standard deviation value of the means (±SD).

FIG. 6 shows the classes related to the different relative phenological parameters such as A) plant height (cm); B) number of leaves, assessed during the cropping season in the experimental field for the agronomic assay on onion var. Elenka. Data are reported as standard deviation of the means (±SD) of the relative recorded values (i.e., plant height (cm) and number of leaves). Means marked by the same letters indicate a not significant difference between the treatment groups. Means marked by different letters indicate a significant difference (One-way ANOVA, Tukey's test, p<0.001) between the treatment groups. Bars indicate the standard deviation values of the means (±SD).

FIG. 7 shows the average values of vigor data recorded during the cropping season for the agronomic test on fennel var. Crono: A) second survey; and B) third survey. Data are reported as means ±SD. Means marked by the same letters indicate a not significant difference (One-way ANOVA, Tukey's test, p>0.05) between the treatment groups. The means marked by different letters indicate a significant difference (One-way ANOVA, Tukey's test, p<0.001) between the treatment groups. The bars indicate the value of the standard deviation of the means (±SD).

### SUMMARY OF THE INVENTION

Object of the present invention is the use of at least one Streptomyces venezuelae strain as a phytopharmaceutical drug, or phytosanitary product, in particular as a fungicide or bio-fungicide, and the use of said strain to trigger the plant immune defenses.

Another object of the present invention is a method of phytosanitary treatment for the control of phytopathogens or eradication of phytopathogens comprising the application on the seed, plant, or soil of a formulation comprising a Streptomyces venezuelae strain; further a method of phytosanitary treatment for the prevention and/or treatment of plant diseases caused by phytopathogens comprising the application on the seed, plant, or soil of a formulation comprising a Streptomyces venezuelae strain. A further object of the invention is a method of phytosanitary treatment to trigger the plant immune defenses comprising the application on the seed, plant, or soil of a formulation comprising a Streptomyces venezuelae strain.

Moreover, the invention relates to formulations comprising a Streptomyces venezuelae strain, as an active ingredient, and at least one acceptable excipient (co-adjuvant and/or co-formulation agent) and/or carrier.

A further object of the invention is a Streptomyces venezuelae strain referred to as DLS1568, deposited at the Leibeniz-Institut DSMZ-Deutsche Sammlung von Mikroorganismen und Zellkulturen GmbH with accession number DSM 33887.

### DETAILED DESCRIPTION OF THE INVENTION

The present invention relates to the use of at least one Streptomyces venezuelae strain, in particular the Streptomyces venezuelae DSM 33887 strain, as a phytopharmaceutical drug, or phytosanitary product, useful in the biological management, i.e., in the control or the eradication, of phytopathogens, in particular fungi, causing plant diseases.

It has been surprisingly found that such microorganisms belonging to the genus Streptomyces species venezuelae, in particular the Streptomyces venezuelae DSM 33887 strain, are able to control or to eradicate a wide range of phytopathogens taxonomically different from each other.

According to the invention, the term “control of phytopathogens”, means a bio-control activity, i.e., of direct antagonism of microbial agents, in particular fungal pathogens, harmful for cultivated plants, particularly those plant for which the seed represents the vegetative propagation organ.

Streptomyces venezuelae is a Gram-positive bacterium belonging to the order Streptomycetales, family Streptomycetaceae, genus Streptomyces, isolated from the microbiota associated to the rice (Oryza sativa L.) seed. Streptomyces venezuelae is an aerobic, filamentous, and spore forming bacterium.

A further object of the invention is a Streptomyces venezuelae strain referred to as DLS1568, which has been deposited at the Leibeniz-Institut DSMZ-Deutsche Sammlung von Mikroorganismen und Zellkulturen GmbH with accession number “DSM 33887”, the 1^{st} of June 2021 under the Budapest Treaty.

According to a preferred aspect, at least one Streptomyces venezuelae strain, in particular the Streptomyces venezuelae DSM 33887 strain, is used as a fungicide, or bio-fungicide, for the control or the eradication of phytopathogens, in particular fungi, affecting and damaging plants of agricultural interest.

The pathogenic fungi are preferably selected from: Fusarium oxysporum f. sp. Cepae (FOC), F. oxysporum f. sp. raphani (FOR), F. oxysporum f. sp. lactucae (FOL), Sclerotinia sp., Pythium sp., Stemphylium botryosum, Colletotrichum dematium.

The present invention relates to formulations comprising a Streptomyces venezuelae strain, preferably a Streptomyces venezuelae DSM 33887 strain, as active ingredient, at least one acceptable excipient and/or co-formulation agent and/or carrier, i.e., compatible with the biology, vitality, and efficacy of the streptomyces.

The compositions can be formulated following conventional methods and are preferably used in liquid form, for example in the form of dispersions or suspensions of the microorganism, or in powdered formulations of the same microorganism.

Preferably, the excipient, in particular in a formulation to be applied on the seed, is Bio-Friendly 1 (Binder/Coating) SEEDWORX™ [Centor Europe, Andijk, The Netherlands], which does not compromise the microorganism vitality, the seed germination, and the seed development.

Streptomyces venezuelae, preferably the Streptomyces venezuelae DSM 33887 strain, can be present in the formulation in a weight ratio to the total weight (w/w) of the composition from 5% to 75%, preferably from 20% to 40%, even more preferably is equal to 30%. Preferably, Streptomyces venezuelae, preferably the Streptomyces venezuelae DSM 33887 strain, can be present in the composition in an amount from 10⁷ to 10⁹, preferably equal to 10⁸ CFU (Colony Forming Units) for 1 g of product.

A further object of the invention is the use of a Streptomyces venezuelae strain, preferably the Streptomyces venezuelae DSM 33887 strain, or formulations thereof, in the prevention and/or treatment of plant diseases caused by phytopathogens, in particular fungi, affecting crops preferably at both plants' collar and canopy.

An object of the invention is a method of phytosanitary treatment for the control of phytopathogens causing plant diseases or a method for the prevention and/or treatment of plant diseases caused by phytopathogens, in particular fungi, comprising the application on the seed, plant, or soil of a formulation comprising a Streptomyces venezuelae strain, preferably the Streptomyces venezuelae DSM 33887 strain.

According to a preferred aspect, the application comprises the seed coating, for example by dry mixing or with a liquid composition, before sowing.

According to another preferred aspect, the application comprises the spraying of the seedlings or, as an alternative, the mixing with the soil substrate before sowing or the cultivation plant as an aqueous solution or the soil treatment immediately after transplanting.

According to another preferred aspect, the application comprises the immersion in an aqueous suspension comprising Streptomyces venezuelae, preferably the DSM 33887 strain, of the plantlets roots or rooted bulbs before transplanting.

Further, the application may comprise the treatment during the stages of crop cultivation as a foliar spray against the pathogenic microorganisms causing diseases, in particular fungal diseases, to the leaves and to the other aerial parts of the plant.

Moreover, in preparation for treatment with Streptomyces venezuelae, preferably with the DSM 33887 strain, the seed can be treated by ozonation, preferably for a period of 45 minutes at 20 ppm of ozone, to significatively reduce the microbial load associated to seed, therefore obtaining both a reduction of the seed microbiota, possibly competitive with the same streptomyces, and an effect stimulating the germination and the development of the seedlings.

The performed experimental assays showed that the main mechanisms of action of the antagonistic microorganism Streptomyces venezuelae, in particular the DSM 33887 strain, in the bio-control of phytopathogens comprise:
1) the direct parasitism action of the pathogen by physical attack and through the secretion of lytic enzymes;
2) the competition for the nutrients required for the pathogen development or its ability to colonize the habitat;
3) the production of secondary metabolites with toxic and inhibitory activity against the phytopathogens (antibiosis).

Moreover, the performed experimental assays showed the internalization in the plant tissue of the Streptomyces venezuelae microorganism, in particular the DSM 33887 strain, and the consequent plant colonization by the microorganism, resulting in plant growth promotion and possible stimulation of the endogenous immune defenses of the plant itself. In the endophytic phase, the microorganism colonies rapidly multiply, colonizing first the host plant roots and then the aerial parts, such as stem and leaves, occupying an important ecological niche at the expense of a possible colonization of the pathogenic microorganisms, which, therefore, loose their invasiveness and pathogenic efficiency. Further, the microorganism stimulates the host plant development, since during the rhizosphere colonization process, the production of growth enhancer enzymes and metabolites occur, which stimulate the root and the aerial part development, and the host plant immune defenses.

From the performed studies it was evident that the streptomycete can be able to induce in planta gene expressions related to a systemic resistance response against pathogens, demonstrating a plant immune response when it is stimulated by the contact with the streptomycete.

A further object of the invention is the use of at least a Streptomyces venezuelae strain, preferably the Streptomyces venezuelae DSM 33887 strain, to enhance the plant immune defenses.

A further aspect of the invention is a method of phytosanitary treatment to enhance the plant immune defenses comprising the application on the seed, plant, or soil of a composition comprising a Streptomyces venezuelae strain.

The following Examples further illustrate the invention.

### EXAMPLES

### Formulation Examples

### Example 1

A formulation for seed tanning was prepared, wherein 20 ml of deionized water at a final concentration of 10⁹ UFC ml⁻¹ of Streptomyces venezuelae DSM 33887 were applied per each Kg of seed, and the co-formulation agent Bio Friendly 1 (Binder/Coating) SEEDWORX™ [Centor Europe, Andijk, The Netherlands] was added in a 1:1 ratio.

### Experimental Examples

The following experimental Examples relate to the evaluation by in vitro test, under controlled environment, and by field trials of the efficacy of the composition according to the invention of Example 1 in the control of phytopathogens and in the prevention, control, and treatment of plant diseases caused by phytopathogens, through the use of Streptomyces venezuelae.

### Materials and Methods

### Microbic Cultures

The bacterial strains used in the present study are present in the collection of the Laboratorio di Patologia Vegetale, Department of Life Sciences, University of Modena and Reggio Emilia (Table 1).

TABLE 1
List of antagonistic bacterial strains used in the present study

| Bacterial | Identification | | Geographic |
|---|---|---|---|
| antagonist | code | Isolation plant matrix | origin |
| Streptomyces sp. | DLS 1568 | Rice (Oryza sativa) | Italy |
| | | seed | |
| Streptomyces sp. | SA 51 | Olive (Olea europaea | Italy |
| | | L.) roots | |
| Pseudomonas sp. | PT 65 | Tomato (Solanum | Italy |
| | | lycopersicum, L.) | |
| | | plants | |
| Agrobacterium sp. | AR 39 | Cob nut (Corylus | Italy |
| | | avellana L.) roots | |

The phytopathogenic fungi used in this study represent the most common agents causing soil diseases affecting horticultural crops (Table 2).

TABLE 2
List of phytopathogenic fungi used in the present study

| | Identification | Isolation plant |
|---|---|---|
| Phytopathogenic fungi | code | matrix |
| Fusarium oxysporum f. sp. cepae | 1585 | Onion |
| Fusarium oxysporum f. sp. raphani | Fus Ruc 9A | Wild rocket |
| Sclerotinia sp. | 1584 | |
| Stemphylium sp. | 1773 | Onion |
| Colletotrichum dematium | 1772 | Spinach |
| Pythium sp. | 1583 | Onion |

### Plant Material

Four different horticultural crops were used during the research (Table 3).

TABLE 3
List of the horticultural species used in the present study

| Scientific name | Common name | Variety | Additional features |
|---|---|---|---|
| Allium cepa | Onion | Fundador | Long day genotype |
| | | Elenka | Long day genotype |
| Foeniculum | Fennel | Crono | Early cycle with |
| vulgare | | | summer sowing |
| Diplotaxis | Wild rocket | CRX 265 (Kristina) | |
| tenuifolia | (Santamaria | CRX 267 (Karina) | |
| | et al., 2002). | Adagio | |
| Lactuca sativa | Lettuce | Summerbel | Blonde Batavia type |

### Example 2—Antagonistic Assay In Vitro

The microbial antagonism was studied using the double plate culture method. Each bacterial culture of Table 1 was cultivated in nutrient liquid broth (NSB=nutrient broth 8.0 gr l⁻¹, saccharose 50 gr l⁻¹) for 24 hours at 27±2° C. A drop (20 μl) of the bacterial suspension was then recovered from the growing culture and applied to a side of a Petri dish, at 1 cm from the edge, containing potato dextrose agar substrate (PDA=potato extract 4 g l⁻¹, dextrose 20 g l⁻¹, agar 15 g l⁻¹) 24 hours before the fungal inoculum. A disc (3 mm) with fungal mycelium grown on PDA for five days was then placed on the opposite side (1 cm from the edge) of each plate, in order to evaluate the bacterial inoculum antagonism. Plates containing the same nutrient substrate, but without the adding of antagonistic bacteria, were used as control. All plates were then incubated at 27±2° C. for seven days. The fungal growth inhibition (IG) was assessed by percentage, calculated as the mean (three experimental replicates for each isolate) of the fungal mycelium diameter formed around the area of the bacterial colony according to the formula reported below (Wang et al., 2011):

$$IG \% = \frac{C - S}{C} \times 100$$

(C=diameter of the positive control, S=sample diameter)

### Results

The antagonistic activity of the following four isolated microorganisms: Streptomyces sp. DLS1568, Streptomyces sp. SA51, Agrobacterium sp. AR 39, and Pseudomonas sp. PT 65 was assessed by in vitro assays against seven phytopathogenic fungi: F. oxysporum f. sp. cepae, F. oxysporum f. sp. raphani, Sclerotinia sp., Stemphylium sp., Colletotrichum dematium, Pythium sp.

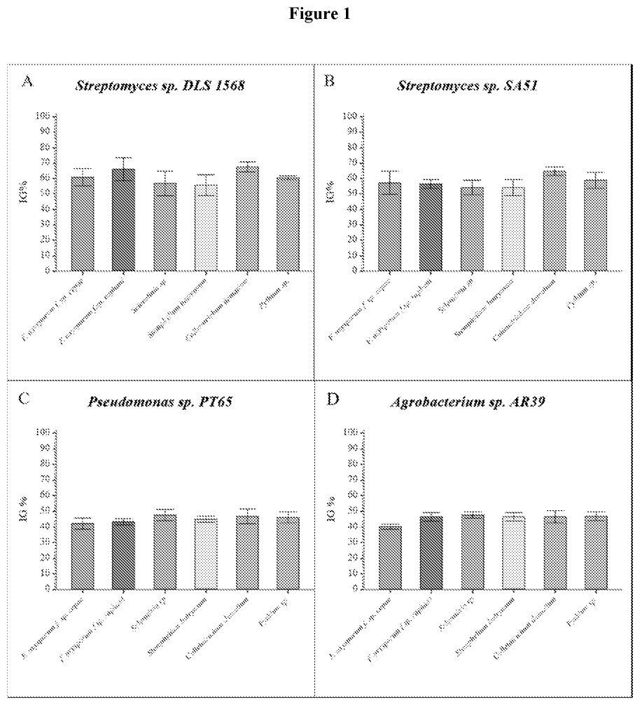

The IG (%) values obtained for each microorganism were reported in the histograms in FIG. 1.

All four microorganisms showed an inhibition activity against the growth of phytopathogenic fungi. However, Streptomyces sp. DLS1568 was particularly active, showing an IG (%) higher than 55% during all the in vitro tests against all seven phytopathogenic fungi (FIG. 1).

Streptomyces sp. DLS1568 further showed the highest inhibition activity against F. oxysporum f. sp. cepae and F. oxysporum f. sp. raphani with an IG (%) up to 60% and 65%, respectively.

Pseudomonas sp. PT 65 and Agrobacterium sp. AR 39 showed a lower inhibition activity (with an IG lower than 50%) against the above-mentioned seven phytopathogenic fungi.

Streptomyces sp. DLS1568 and Streptomyces sp. SA51 showed an IG higher than 50% against all seven phytopathogenic fungi analyzed using the double plate culture method.

### Example 3—Plant Growth Promotion Mechanisms

Two plant growth promotion mechanisms, such as the production of siderophores and the phosphate solubilization activity, were verified for both Streptomyces sp. DLS 1568 and Streptomyces sp. SA51 strains by in vitro assays on agar substrate plates.

### Production of Siderophores

To assess the production of siderophores, the modified Schwyn and Neilands' method (1987) was applied. Both Streptomyces spp. were cultivated in a flask containing the liquid substrate “International Streptomyces Project-2” (ISP-2, yeast extract 4.0 g l⁻¹ malt extract 10.0 g l⁻¹, dextrose 4.0 g l⁻¹) for 48 hours at 26±2° C. The streptomyces microbial suspension was then centrifuged at 6000 rpm for 20 minutes, and the resulting pellet was preliminarily washed and then re-suspended in sterile distilled water; an aliquot of such suspension was inoculated in agar CAS-LB (agar substrate LB added with Chrome Azurol S) prepared according to Schwyn and Neilands (1997). Sterile distilled water was used as negative control and, a 1M pyrocatechol solution (Lakshmanan et al., 2015) was used as positive control. The plates were then incubated at 28±2° C. After a 5 days incubation, the development of a yellow-orange halo was measured, which indicates the production of siderophores. Three experimental replicates per strain were carried out. Detected data were subjected to statistical analysis of variance (one-way ANOVA). The results were compared to the post hoc Tukey's HDS test.

### Results

The microorganisms can have the feature to produce and secrete siderophores adapted to sequestrate and metabolize the iron present in the rhizosphere. Both the isolated strains: Streptomyces sp. DLS 1568 and Streptomyces sp. SA51 showed the ability to produce siderophores, as highlighted from the colour changing in the blue CAS substrate from green-bluish to orange.

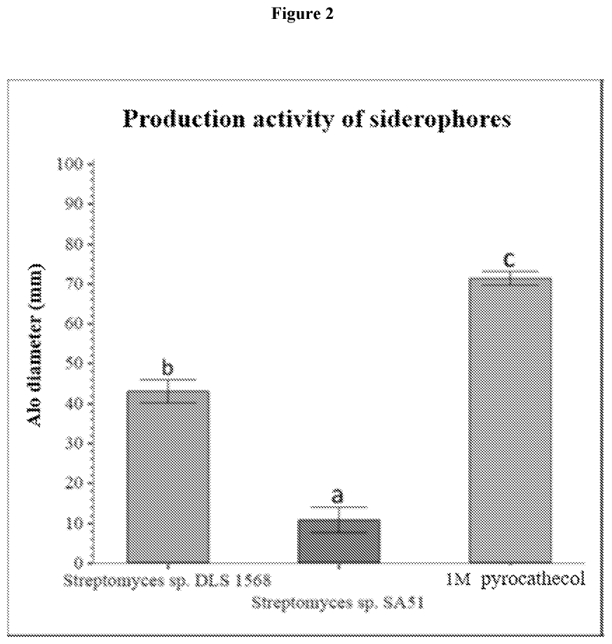

The difference in the production of siderophores was assessed by measuring the diameter of the developed orange halo. Streptomyces sp. DLS 1568 showed a greater production of siderophores, by recording an average value of halo diameter equal to 43 mm (SD=5.00) with respect to the average value of 11 mm (SD=5.29) of Streptomyces sp. SA51. The statistical analysis displayed a significant difference (p<0.001) between the average diameter values of the yellow halo (mm) for the Streptomyces sp. DLS 1568, Streptomyces sp. SA51 strains and the positive control (1M pyrocatechol) (FIG. 2).

### Phosphate Solubilization

The Streptomyces sp. DLS1568 and Streptomyces sp. SA51 strains were studied for their ability of phosphate solubilization, using the modified Lucas et al. (2014) protocol. A drop of streptomyces bacterial suspension (20 μl) was placed in the center of the Petri dish with agar substrate. A drop of sterile distilled water (20 μl) was used as negative control. After 14 days incubation, the ability of phosphate solubilization was assessed through the assessment of the presence and the measurement of the diameter of a zone (the so-called halo) of a lighter colour around the colony of the Streptomyces sp. strains. Three technical replicates per strain were carried out. Recorded data were subjected to statistical analysis of variance (one-way ANOVA). The results were compared to the post hoc Tukey's HDS test.

### Results

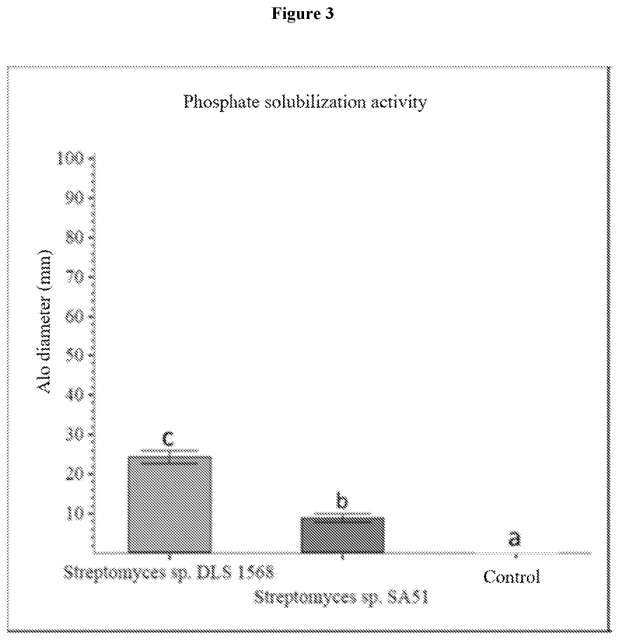

The Streptomyces sp. DLS 1568 and Streptomyces sp. SA51 strains were assayed for their ability to metabolize phosphate by evaluating their ability of extracellular solubilization of the tricalcium phosphate precipitated with glucose as the sole carbon source. The Streptomyces sp. DLS 1568 strain showed a higher and statistically significant (p<0.001) activity of mineral phosphate solubilization, displaying an average value of the lighter colour balo of 24.33 mm (SD=3.05) compared to the Streptomyces sp. SA51 strain, who showed an average value of 9.00 mm (SD=2.00) (FIG. 3).

### Example 4—Observation at the Confocal Laser Scanning Microscope (CLSM) of Endophytic Colonization in Planta by EGFP-Streptomyces sp. DLS 1568

The onion, fennel, wild rocket, and lettuce seeds, respectively, were used for the microbial seed coating with the mutant EGFP-Streptomyces sp. DLS1568 strain, transformed with the GFP protein (Green Fluorescent Protein) in order to make it traceable in the plant tissue during its in-planta internalization and colonization. Each seed sample (n=1 g of seed) was surface disinfected according to Fatmi et al. (1991). Briefly, seeds were treated in 2% sodium hypochlorite (NaOCl) for 20 minutes at 25° C. on a rotary shaker at 250 rpm (Innova, New Brunswick Scientific, Edison, NJ and then rinsed under running tap water for three minutes.

The streptomyces bacterial suspension used for the seed coating was grown into liquid medium ISP-2 for 24 hours at 27±2° C. The suspension was then centrifuged at 6000 rpm for 20 minutes. The supernatant was discarded and the pellet resuspended in 20 ml of sterile distilled water. The bacterial concentration was determined using a spectrophotometer (Spectronic 20; Bausch and Lomb, Rochester, NY) (optical density at 600 nm=0.3; about 1×10⁸ CFU ml⁻¹) and calibrated at a final concentration of 10⁸ CFU ml⁻¹.

The seed samples were treated with sterile distilled water (used as negative control) and with the above-described bacterial suspension, according to Kaufman (1991), respectively. Briefly, each seed sample was incubated 20 minutes at 25° C. on a rotary shaker at 250 rpm (Innova, New Brunswick Scientific, Edison, NJ), air dried for 24 hours at room temperature and then stored at 4° C. until sowing. The assessment of the internalization of the Streptomyces sp. DLS 1568 strain was performed on seedlings grown 20 days after the sowing. For each sample, roots, stems, and leaves were carefully dissected (approximately 10 mm) with a sterile scalpel and placed on the slide for observation through a confocal microscope (CLSM) Leica TCS SP8 (Leica Microsystems, Mannheim, Germy), equipped with a solid state laser able to excite the fluorescence of the GFP protein of the mutant EGFP-Streptomyces sp. DLS1568 strain, thus allowing to verify the presence and the possible colonization of the plant tissues. The laser scanning microscopy analysis was conducted at CIGS (Centro Interdipartimentale Grandi Strumenti, University of Modena and Reggio Emilia, Italy)

### Results

The Streptomyces DLS 1568 internalization was studied by confocal laser scanning microscopy (CLSM) using different plant tissues, such as roots and leaves of onion, fennel, wild rocket, and lettuce from seedlings developed 3 weeks after sowing. The endophytic colonization of plant tissues by EGFP-Streptomyces sp. DLS 1568 was observed in 99% of the tissue sections of all the seedling samples. The abundant colonization by EGFP-Streptomyces sp. DLS 1568 demonstrated the capability of this microorganism to interact with the host from the early stages of seed germination and during the development of roots and leaves. The cells of the mutant EGFP-Streptomyces sp. DLS 1568 showed a uniform distribution among all various plant tissues. These results confirm that Streptomyces sp. DLS1568 can internalize and colonize the plant tissues, highlighting the long-term interaction potential with all the four different crops assessed in this study.

### Example 5—Germination Test

Samples of onion, fennel, wild arugula, and lettuce seeds, were respectively coated with Streptomyces sp. DLS 1568 (see Example 2 for treatment specifications) and through experimental ozonation treatment, with the respective uncoated controls, were assayed for their germination performances. Each seed sample (n=100 seeds) was assayed in four replicates, according to the ISTA protocol (International Seed Testing Association, 2018) for the respective plant species. The seed samples were placed on germination test paper (Sartorius, Göttingen, DE), moistened, and subsequently placed in plastic boxes. The plastic boxes were then placed in the germination chambers at an average temperature of 21±2° C., with 16 hours of daylight. After the incubation period, the number of germinated seeds was counted for each box and the germination percentage on the total tested seeds was calculated. A seed was considered germinated when the seedling and all the essential structures thereof such as stem, epicotyl, cotyledons, bypocotyl, and rootlets were well developed, complete, proportionated, and healthy, according to the Handbook on Seedling Evaluation (International Seed Testing Association, 2018). The germination test result was expressed in percentage, according to the herein below formula, considering the number of normal, abnormal seedlings, hard, fresh, and dead seeds. Four technical replicates were carried out for each sample.

$$Germination percentage \% = \frac{n}{N} \times 100$$

Wherein n represents the number of germinated seeds and N represents the total number of seeds used for the test.

The seed samples of onion var Elenka and fennel var. Crono, obtained from the seed coating through the previously described process in Example 2 with Streptomyces sp. SA51 and by the application of Bio-Friendly 1 (Binder/Coating) SEEDWORX™ [Centor Europe, Andijk, The Netherlands], were included as commercial controls. The recorded data were subjected to statistical analysis of the variance (one-way ANOVA). The results were compared to the post hoc Tukey's HDS test.

### Results

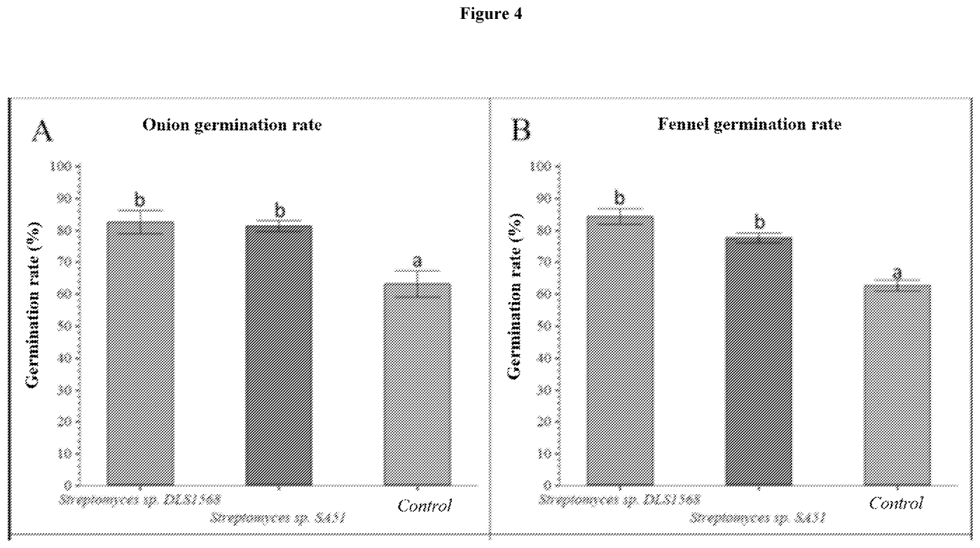

Germination data obtained for the coated seed with Streptomyces sp. DLS1568 and Streptomyces sp. SA51 showed an increase of the germination performance of the respective samples, with respect to the uncoated controls. For the onion (FIG. 4A), the registered germination percentage for the uncoated seeds (negative control) was 65%, while for both treatments with Streptomyces sp. DLS1568 and Streptomyces sp. SA51 a significant increase of the germination was assessed, up to 20% (p<0.05). Similar results were obtained during the fennel germination test. FIG. 4B showed that for the treatments with Streptomyces sp. DLS1568 and Streptomyces sp. SA51 the germination increased up to 30% compared to the uncoated control. Further, Streptomyces sp. DLS1568 showed germination values higher than Streptomyces sp. SA51 in both tests (i.e., onion and fennel), even if the difference was not significant (p>0.05).

### Example 6—In Planta Experiments under Controlled Conditions to Assess the Efficacy of Microbial Coating on Wild Rocket as Bio-Control Operation of the Tracheofusarium Disease (FOR)

On wild rocket var. Adagio, susceptible to Fusarium oxysporum f. sp. raphani (FOR), an experimental assay was carried out to evaluate the efficacy of Streptomyces sp. DLS1568, as a bio-control agent applied by seed coating, against the phytopathogenic fungus FOR (Fus Ruc 9A strain), according to the experimental protocol of Gilardi et al. (2007). The experimental assay was carried out under controlled conditions (phytotron) on wild rocket var. Adagio seed samples treated with Streptomyces sp. DLS 1568 and Streptomyces sp. SA51 (commercial control), following the protocol described in Example 2. Samples of uncoated seeds were used as control.

For the preparation of the fungal inoculum, the conidial suspension of FOR was prepared adding 15 ml of sterile distilled water to rehydrate the fungal conidia at the respective PDA plates wherein the phytopathogenic fungus has grown in incubation for 7 days. Later, the conidial suspension was filtered with a gauze. The conidial concentration was then determined by the use of a hemocytometer and calibrated with deionized water to a final concentration of 10⁷ CFU ml⁻¹, subsequently applied as inoculum in all trials.

The seeds were then sown in polystyrene trays, for a total of 104 seeds per track. Twenty days after sowing, the seedling roots were cut at a 5 cm length and placed for 20 minutes into a calibrated conidial suspension of the fungal phytopathogenic strain FOR (Fus Ruc 9A), as previously described. Twenty-five treated seedlings for each track were transplanted in new trays. Each tray (n=25) was considered as an experimental replicate, for a total of 4 replicates per treatment. The disease severity index values (Severity) are reported in Table 4. The recorded data of disease severity were subjected to statistical analysis of the variance (one-way ANOVA). The results were compared to the post hoc Tukey's HDS test.

TABLE 4
Phytopathometric scale showing the disease severity index with
values comprised between 0 (healthy plant) and 100 (dead plant).
Disease severity index

| | |
|---|---|
| 0 | Healthy plant |
| 12.5 | Plant with light vascular discolorations |
| 25 | Plant with a reduced growth, vascular discolorations, and |
| | an initial foliar chlorosis |
| 50 | Plant with a reduced growth, vascular discoloration, and |
| | foliar chlorosis |
| 75 | Plant with remarkable growth reduction, wide vascular |
| | discolorations, remarkable foliar chlorosis, and necrosis |
| 100 | Dead plant |

### Results

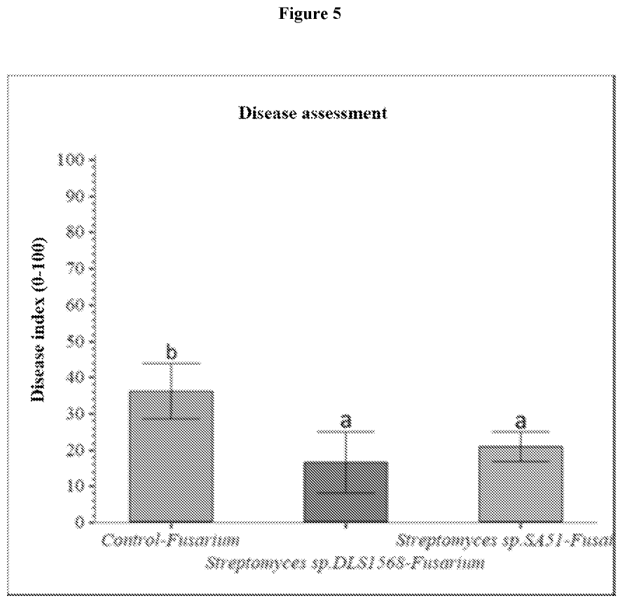

The experimental assay carried out under controlled conditions (phytotron), showed that the plantlets developed from seed coated with Streptomyces sp. DLS 1568 and Streptomyces sp. SA51 showed a mean disease severity index caused by the infection from the pathogenic strain FOR (Fus Ruc 9A) significatively lower (p<0.05) than the uncoated seed sample (control). For the uncoated wild rocket plants (control), a mean disease severity index of 36.25% was recorded. The seed coating treatments with both the isolated strains Streptomyces sp. DLS 1568 and Streptomyces sp. SA51 showed a significant protection (p<0.05) against Fusarium wilt disease. From the seed coated with the isolate Streptomyces sp. DLS 1568, the highest bio-control action/efficacy was recorded, showing a relative average disease severity index of 16.50%, even if not significatively different (p>0.05) than the seed coated with Streptomyces sp. SA51, that showed a mean disease severity index of 20.75% (FIG. 5).

### Example 7—Field Trials with Onion var. Elenka to Assess Germination Performances and Bio-Control Activities under Natural Fusarium spp. Inoculum Pressure (FC2020)

In this experiment, onion var. Elenka was tested. The respective tanning and ozonation seed treatments are listed in Table 5. The test experimental design was organized at randomized blocks for the 4 treatment types, each in duplicate. In the extended test carried out at an experimental field with high natural pressure of fungal inoculum of Fusarium spp., the experimental plots had the following dimensions: 1.8 m (width)×25 m (length) with four sowing rows per parcel The sowing was performed with mechanical seeder (mod. SPX 2015-Bassi Seminatrici, IT) at the density of 600,000 onion seeds per hectare. The surveys were carried out on a length of 20 meters, in the medial part of each respective plot. The recorded data of disease severity were subjected to statistical analysis of the variance (one-way ANOVA). The results were compared to the post hoc Tukey's HDS test.

In the preparation phase before the sowing, the fields were not treated with herbicides or fungicides. During the experimental test, carried out following the agronomical good practices, harmful bacteria and insects' infections were not observed, causing damages to the cultures during the experiment. The weeds were controlled according to the disciplinary regulation described in Cornell Vegetable Management Guidelines and Recommendations (Reiners et al., 2019).

Samples of symptomatic material such as onion roots and bulbs were withdrawn during the cultural season for isolating Fusarium-like fungal colonies and the subsequent identification thereof and confirmation of the etiological agent, through a preliminary analysis of conidial identification at the microscope, followed by a molecular analysis. The molecular identification was carried out through the amplification of the conserved region ITS by polymerase chain reaction (PCR) according to White et al., 1990. The amplification products were then purified and sequenced at Bio-Fab Research s.r.l. (Rome, IT). The nucleotide sequences of ribosomal DNA of the conserved region ITS (Internal Transcription Spaces) were then analyzed through the program for the research of sequence similarity in the international DNA database BLAST (Basic Local Alignment Search Tool;//www.ncbi.nlm.nih.gov/BLAST).

TABLE 5
List of the treatments on onion seed studied during
the experimental field test in Cesena (FC)
Treatment

| | |
|---|---|
| 1 | Control (uncoated seed) |
| 2 | FLUDIOXONIL [0.3 ml Kg⁻¹ seed; treatment according to the |
| | company treatment practices] |
| 3 | Streptomyces sp. SA51 [BINDER: 20 gr Kg⁻¹ seed + Bacterial |
| | suspension: 20 ml * Kg⁻¹ seed] |
| 4 | Streptomyces sp. DLS1568 [BINDER: 20 gr Kg⁻¹ seed + Bacterial |
| | suspension: 20 ml * Kg⁻¹ seed] |
| 5 | Ozone (seed ozonation treatment) |

Note:
* bacterial suspension concentration: 1 × 10⁹ CFU ml⁻¹

### Results

In the experimental test carried out during the first survey (18 days after the sowing), the number of the emerged seedlings was assessed according to the above-described procedure. The seedlings generated from the seeds treated with Streptomyces sp. DLS 1568 (210.75; SD=36.71) and Ozone (T3) (214.63; SD=53.4) showed an increase of the germinal emergence of 15% and 17%, respectively, with respect to the control (182.50; SD=26.22). Such seedlings (generated from seeds treated with Streptomyces sp. DLS 1568 and Ozone (T3)) showed an increase of the germinal emergence of 20% and 22%, respectively, with respect to the treatment with fludioxonil (175.63; SD=32.70). For such treatments (i.e., Streptomyces sp. DLS 1568 and Ozone (T3)), an increase of the germinal emergence of 24% and 26%, respectively, with respect to the treatment with Streptomyces sp. SA51 (169.13; SD=25.82) was registered. However, the statistical analysis of the variance (One-way ANOVA, Tukey's test) did not show significative differences (p>0.05).

During the second survey (42 days after the sowing), the number of plants emerged from seeds treated with Ozone (207.88; SD=35.62) was significantly higher (p <0.05) of 41% and 51% with respect to the treatments with fludioxonil (147.00; SD=41.52) and Streptomyces sp. SA51 (137.71; DS=18.25). Furthermore, the number of plants emerged from seeds treated with Streptomyces sp. DLS 1568 (198.13; SD=43.53) showed a significant increase of 44% (p<0.05) and 34% (p<0.05), respectively, with respect to the treatment with Streptomyces sp. SA51 and Fludioxonil.

During the third survey (70 days after the sowing) a tendency of germination performance in line with the preceding 2 surveys was found, in particular showing the better germination performance for the above-mentioned various treatments. Finally, during the fourth survey (97 days after the sowing), the data related to the number of emerged plants showed that for both treatments Streptomyces sp. DLS 1568 (137.40; SD=16.41) and Ozone (119.17; SD=19.24) a higher number of plants with respect to the treatments carried out through the application of fludioxonil (72.75; SD=8.10) and Streptomyces sp. SA51 (87.17; SD=10.57) was registered. In particular, the number of plants developed and present in cultivation from seeds treated with Streptomyces sp. DLS 1568 (198.13; SD=43.53) showed a significant increase of 57% (p<0.05) and 72% (p<0.05), respectively, with respect to the treatment with Streptomyces sp. SA51 and Fludioxonil.

Through the analysis of the symptomatic samples of onion plants, collected during the experimental field assay, the presence of a high natural inoculum pressure was confirmed through the isolation and identification of Fusarium oxusporum, identified by morphological exam of the fungal isolates (colony features and microscope observation of fruiting bodies, phialides, absence of chlamydospores, etc.) and sequence similarity between 98.50% and 100% of the conserved ribosomal region internal transcribed spacer (ITS) through the comparison with the sequences of the database of the National Center for Biotechnology Information (NCBI) using BLAST.

The higher number of emerged, germinated, and then developed plants recorded during the four surveys, in particular compared to the fungicide treatment with fludioxonil, highlighted the bio-control activity of Streptomyces sp. DLS 1568 through the seed coating against the phytopathogenic fungi Fusarium oxysporum.

### Example 8—Experimental Field Trials with Onion var. Elenka for the Assessment of the Agronomic Features (Phenological Development) (FC2020)

For the assay description, experimental design, agronomic practices, see Example 7. Before sowing, the fields were not treated with herbicides or fungicides. During the experimental field trial, carried out according to the best agronomical practices, harmful bacteria and insects' infections were not observed, causing damages to the cultures during the experiment. The weeds were managed according to the disciplinary regulations described in Cornell Vegetable Management Guidelines and Recommendations (Reiners et al., 2019).

The registered data were subjected to the statistical analysis of the variance (one-way ANOVA). The results were compared to the post hoc Tukey's HDS test.

### Results

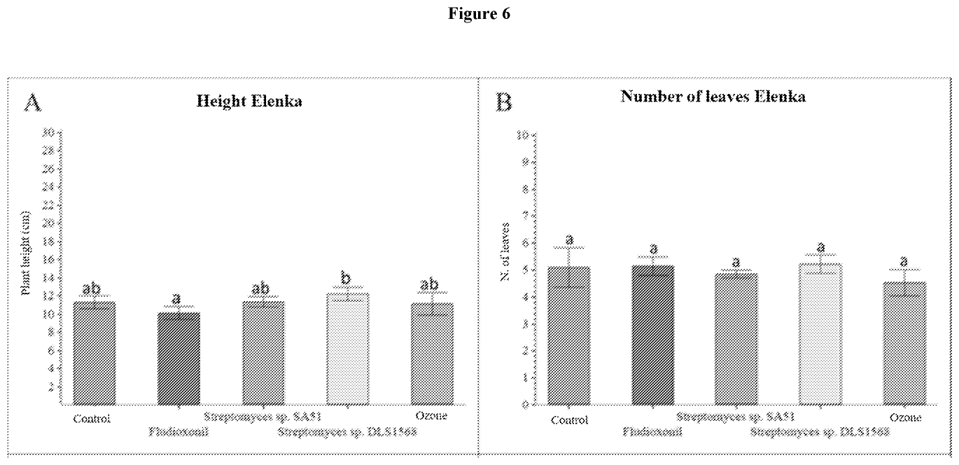

As shown in FIG. 6A in the assessment of the plant height (second survey), the treatment with Streptomyces DLS 1568 showed a significant increase of the plant height (12.23 cm; SD=0.9) compared to fludioxonil (10.11 cm; SD=0.7; p<0.05). The assessment of the number of leaves (third survey) for the plants developed for the 5 tracks, the registered values did not point out significative differences (p>0.05) among the various tracks, despite the tracks with seeds treated with Streptomyces DLS 1568 (5 23; SD=0.2) and Ozone (4.69; SD=0.6) registered the highest values (FIG. 6B).

### Example 9—Field Trials with Onion var. Fundador for the Assessment of the Germination Performance and Bio-Control Action at Experimental Field with Natural Pressure of Inoculum of Fusarium spp. (FC2020)

For trial description, experimental design, agronomic practices, see Example 7. In this experiment, onion var. Fundador was used, applying the seed treatments listed in Table 5. The recorded data were subjected to statistical analysis of the variance (one-way ANOVA). The results were compared to the post hoc Tukey's HDS test.

### Results

During the first survey (18 days after the sowing), the number of plants emerged from seeds treated with Streptomyces sp. DLS 1568 (212.87; SD=33.99) was significatively higher (p<0.05) of 20%, 51% and 15%, compared to the control (178.12; SD=30.92), Streptomyces sp. SA51 (141.12; SD=35.71) and Ozone treatment (185.87; SD=33.24), respectively. No significative difference (p>0.05) was observed between Streptomyces sp. DLS 1568 and with Fludioxonil treatment (210.12; SD=57.65). During the fourth survey, the number of plants emerged from seeds treated with Streptomyces sp. DLS 1568 (154.57; SD=16.38) was significatively higher (p<0.05) compared to the treatment with Streptomyces sp. SA51 (103.50, SD=35.61). From the isolation and the identification of symptomatic plant material, the presence and the identity of phytopathogenic Fusarium oxysporum fungal isolates was confirmed, confirming the presence of a high pressure of natural inoculum (see Example 7).

The number of emerged, germinated, and then developed plants recorded during the four surveys, from seed coated with Streptomyces sp. DLS 1568, highlighted an increase of the recorded values compared to Streptomyces sp. SA51. Further, these values are in line with the seeding fungicide treatment with fludioxonil, thus highlighting the bio-control action performed by Streptomyces sp. DLS 1568 through the seed coating against the pathogenic fungi Fusarium oxysporum.

### Example 10—Trials in Commercial Fields with Fennel var. Crono for the Assessment of the Emergence and Germination Performances (AQ2020)

In this experiment, the fennel var. Crono was used, in order to study the effect of Streptomyces sp. DLS 1568, applied as microbial coating agent, on the emergence and germination of the crop. The respective seed treatments are listed in Table 6.

The test experimental design was organized at randomized blocks for the 4 treatment types, each in duplicate (replicate). In the experimental test carried out at a plot, the experimental parcels had the following dimensions: 1.5 m (width)×100 m (length) with four sowing rows per plot. The sowing was carried out with a mechanical seeder. During the experimental field trials, conducted according to the best agronomical practices, harmful bacteria and insects' infections were not observed, causing damages to the cultures during the experiment. Weeds were controlled according to the farms' integrated weed management.

The first survey was carried out 20 days after the sowing, assessing the whole length of each plots. The second survey was carried out 32 days after seed sowing and the third and last survey was done after one month from the second survey. The recorded data were subjected to the statistical analysis of the variance (one-way ANOVA). The results were compared to the post hoc Tukey's HDS test.

TABLE 6
List of the seed treatments carried out on fennel
var. Crono used during the field trials (AQ).
Treatment

| | |
|---|---|
| 1 | Control (uncoated seed) |
| 2 | FLUDIOXONIL [0.3 ml Kg⁻¹ seed; treatment according to the |
| | company treatment practices] |
| 3 | Streptomyces sp. DLS1568 [BINDER: 20 gr Kg⁻¹ seed + bacterial |
| | suspension: 20 ml * Kg⁻¹ seed] |

Note:
* bacterial suspension concentration: 1 × 10⁹ CFU ml⁻¹

### Results

The emergence and germination values recorded overall for the treatment with Streptomyces sp. DLS1568 showed an increase of the germination performances and of the number developed healthy plantlets compared to control and fludioxonil treatments (Table 7). During the first survey for the number of emerged seedlings, the treatment with Streptomyces sp. DLS 1568 showed a higher average value, but not significatively higher (p>0.05), of 365.25 (SD=26.79) with respect to the tracks related to the control (339.50; SD=9.33) and fludioxonil (339.50; SD=45.35). During the second survey (after the agronomic practice of manual thinning of the plantlets, usually applied to weak/abnormal plantlets by agricultural operators, leaving on the row an average of 20-25 centimeters of distance between one and the other) the data collected and statistically analyzed showed a significant increase (p<0.05) of the number of emerged plantlets for the seeds treated with Streptomyces sp. DLS 1568 (227.94; SD=8.31) with respect to the control (194.40; SD=14.85). Although the value for the track treated with fludioxonil was lower (217.30; SD=3.89) than the treatment with Streptomyces sp. DLS 1568, the difference was not significant (p>0.05). During the third survey, the data collected and statistically analyzed showed a significant increase (p<0.05) of the number of plants belonging to the treatment with Streptomyces sp. DLS 1568, registering an average value of 212.50 (SD=16.26) with respect to the control track (187.00; SD=7.07) and Fludioxonil (198.62; SD=0.88) (Table 7).

TABLE 7
Number of emerged plants of fennel var. Crono recorded during the
extended plot field trial (AQ2020). Means with same letters indicate a
not significative difference (One-way ANOVA, Tukey's test, p > 0.05)
between the treatment groups. Means with different letters indicate a
significative difference (One-way ANOVA, Tukey's test, p < 0.05)
between the treatment groups. Bars indicate the value of the standard
deviation of the means (±SD).
Fennel var. Crono

| | Survey 1 | | Survey 2 | | Survey 3 | |
|---|---|---|---|---|---|---|
| | Average | | Average | | Average | |
| Treatment | value | SD | value | SD | value | SD |
| Control | 339.50 | ±9.33 ^{a} | 194.40 | ±14.85 ^{a} | 187.00 | ±7.07 ^{a} |
| Fludioxonil | 339.50 | ±45.35 ^{a} | 217.30 | ±3.89 ^{ab} | 198.62 | ±0.88 ^{ab} |
| Streptomyces | 365.25 | ±26.79 ^{a} | 227.94 | ±8.31^{b} | 212.50 | ±16.26^{b} |
| sp. DLS1568 | | | | | | |

### Example 11—Experimental Field Trials with Fennel var. Crono for the Assessment of Emergence, Germination, and Vigor at Plot (AQ2021)

For the field trial description, experimental design, and the applied agronomic practices, see Example 10.

For this experiment, sowing was carried out with mechanical seeder. The first survey was carried out 14 days after the sowing, assessing the whole length of each plot: 1.5 m (width)×100 m (length) with four sowing rows per plot. The second survey was carried out one month after sowing and the third and last survey two months after sowing, assessing both number of emerged plants and vigor according to the assessment scale described in Table 8. The recorded data were subjected to the statistical analysis of the variance (one-way ANOVA). The results were compared to the post hoc Tukey's HDS test.

TABLE 8
Vigor assessment scale for the trials in commercial
field of fennel var. Crono (AQ2021)
Vigor assessment index

| | |
|---|---|
| 1 | Initial phase of development of the first real leaves |
| 2 | Development of the first real leaves |
| 3 | Complete development of the first real leaves |
| 4 | Initial phase of development of the second real leaves |

The data of the emergence for the track related to the treatment of seed tanning with Streptomyces sp. DLS1568 showed an increase of the germination performance and the development of healthy plantlets with respect to the treatments related to control and fludioxonil (Table 9). During the first survey, relating to the number of emerged seedlings, the registered average value for the track related to the coating treatment with Streptomyces sp. DLS 1568 showed a higher average value, but not significant (p>0.05), of 335.13 (SD=6.89) with respect to the coating treatments related to the uncoated control (313.00; SD=4.60) and fludioxonil (323.88; SD=6.19). During the second survey for the assessment of the emerged plants (after the agronomic practice of manual thinning of the plantlets, usually applied to weak/abnormal plantlets by agricultural operators) the data collected and statistically analyzed showed a significant increase (p<0.05) of the number of plantlets from seeds treated with Streptomyces sp. DLS 1568 (209.13; SD=8.31) with respect to the control (180.75; SD=14.85). No significant difference (p>0.05) between the tracks of seed coated with Streptomyces sp. DLS 1568 and Fludioxonil (205.00; SD=3.89) was highlighted for the number of present plantlets. During the third survey, the data collected and statistically analyzed showed a significant increase (p<0.05) of the number of plants belonging to the treatment with Streptomyces sp. DLS 1568 (194.50; SD=12.24) with respect to the uncoated control (173.00; SD=8.05) and the track of the coating treatment with fludioxonil (186.63; SD=1.98).

TABLE 9
Number of emerged plants of fennel var. Crono detected during the
extended plot field trial (AQ2021). Means with same letters indicate a
not significant difference (One-way ANOVA, Tukey's test, p > 0.05)
between the treatment groups. Means with different letters indicate a
significant difference (One-way ANOVA, Tukey's test, p < 0.05)
between the treatment groups. Bars indicate the value of the standard
deviation of the means (±SD).
Fennel var. Crono

| | Survey 1 | | Survey 2 | | Survey 3 | |
|---|---|---|---|---|---|---|
| | Average | | Average | | Average | |
| Treatment | value | SD | value | SD | value | SD |
| Control | 313.00 | ±4.60 ^{a} | 180.75 | ±14.85 ^{a} | 173.00 | ±8.05 ^{a} |
| Fludioxonil | 323.88 | ±6.19 ^{a} | 205.00 | ±3.89 ^{b} | 186.63 | ±1.98 ^{ab} |
| Streptomyces | 335.13 | ±6.89 ^{a} | 209.13 | ±8.31 ^{b} | 194.50 | ±12.24 ^{b} |
| sp. DLS1568 | | | | | | |

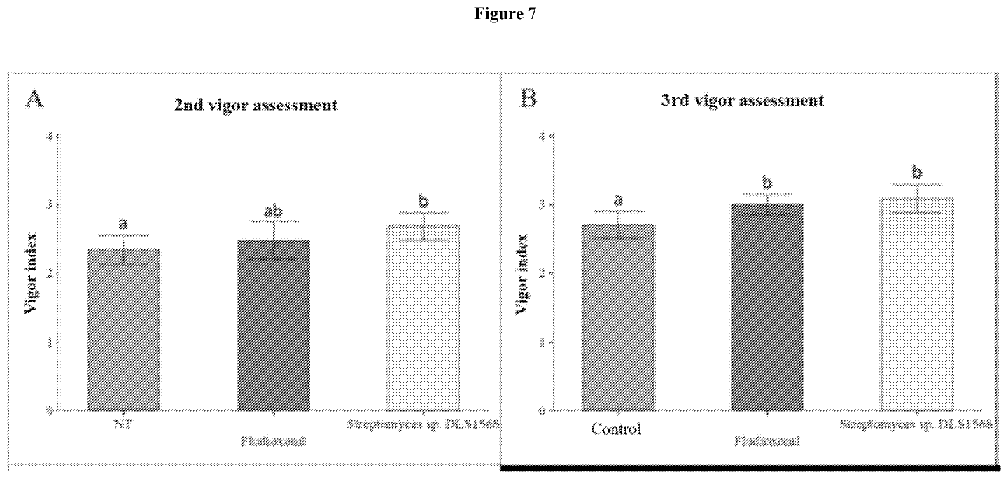

During the second survey wherein the plantlets vigor was assessed, the registered data showed a significant increase (p<0.05) of the average vigor index for the plants germinated from seeds treated with Streptomyces sp. DLS 1568, showing an average value of 2.69 (SD=0.20) with respect to the coating treatment with Fludioxonil (2.49; SD=0.27) and the uncoated control (2.34; SD=0.22) (FIG. 7A). Even for the third survey the data collected and statistically analyzed showed a significant increase (p<0.05) of the average vigor index for the plants germinated from seeds treated with Streptomyces sp. DLS 1568, registering an average value of 3.09 (SD=0.21) with respect to the uncoated control, showing an average value of 2.71 (SD=0.19) (FIG. 7B). No significant difference was pointed out between the tanning treatment with Streptomyces sp. DLS 1568 and fludioxonil (3.01; SD=0.15).

### Example 12—In Planta Detection of Streptomyces sp. DLS 1568

During the field trials carried out for the onion and fennel crops as reported in Examples 7, 8, 9, 10, and 11, the respective plant samples belonging to the seeds treated with Streptomyces spp. DLS 1568 were collected in different phenological stages (i.e., seedlings, plantlets, and plants) to detect and confirm the presence and colonization of this microorganism within the plant tissues. The plants germinated from uncoated seeds were used as negative control. The plant material was collected and subjected to DNA extraction using the DNeasy® Plant Mini Kit (Qiagen, Hilden, Germany). A 2 μl DNA aliquot was then subjected to PCR molecular test (polymerase chain reaction) using the primers specific for the gene 16S rRNA for the genus Streptomyces: StrepB (forward) (5′-ACAAGCCCTGGAAACGGGT-3′) (SEQ ID N. 1) and StrepF (reverse) (5′-ACGTGTGCAGCCCAAGACA-3′) (SEQ ID N. 2), according to the experimental protocol reported in Rintala et al. (2001). The gene amplification (PCR) was performed in a thermal cycler (Applied Biosystems 9700) in 25 μl amplification reactions. After amplification, the samples were then analyzed through electrophoretic ran in 2% agarose gel, stained with ethidium bromide for 30 minutes, and the images were kept with BioDoc Analyze (Biometra, Göttingen, Germany).

### Results

The 93% and 89% of the onion and fennel plant samples, respectively, gave positive result to the specific gene amplification with the primer pairs of the gene rRNA 16S StrepB/StrepF by generating the 507 bp specific fragment. Thus, the ability of Streptomyces sp. DLS1568 to colonize the onion and fennel plant tissues by the seed coating treatment with such microorganism was confirmed. No specific amplification was registered for the uncoated controls.

1. (canceled)

2. The method according to claim 5, wherein the strain is Streptomyces venezuelae DSM 33887.

3. (canceled)

4. (canceled)

5. A method of preventing and/or treating plant diseases caused by phytopathogens by using at least one Streptomyces venezuelae strain.

6. The method according to claim 5, wherein the phytopathogens are pathogenic fungi causing plant diseases.

7. The method according to claim 6, wherein the fungi are selected from: Fusarium oxysporum f. sp. cepae (FOC), Fusarium oxysporum f. sp. raphani (FOR), Fusarium oxysporum f. sp. lactucae (FOL), Sclerotinia sp., Pythium sp., Stemphylium botryosum, Colletotrichum dematium.

8. A method of phytosanitary treatment for the control of phytopathogens or the eradication of phytopathogens comprising the application on the seed, plant, or soil of a composition comprising a Streptomyces venezuelae strain.

9. A phytosanitary treatment method for the prevention and/or treatment of plant diseases caused by phytopathogens comprising the application on the seed, plant, or soil of a composition comprising a Streptomyces venezuelae strain.

10. A phytosanitary treatment method to enhance the plant immune defenses comprising the application on the seed, plant, or soil of a composition comprising a Streptomyces venezuelae strain.

11. The method according to claim 8, wherein the strain is Streptomyces venezuelae DSM 33887.

12. The method according to claim 8, wherein the application comprises the seed coating.

13. The method according to claim 8, wherein the application comprises the spraying of seedlings, or as an alternative, the mixing with the culture substrate before the sowing or the cultivation plant as an aqueous solution or the soil treatment immediately after the transplant.

14. The method according to claim 8, wherein the application comprises the immersion in an aqueous suspension of the plantlets' roots or the rooted bulbs before the transplant.

15. The method according to claim 8, wherein the application comprises the treatment as a foliar spray during the cultivation phases.

16. The method according to claim 8, wherein the application further comprises the seed ozonation.

17. Formulations comprising a Streptomyces venezuelae strain, as active ingredient, and at least one acceptable co-adjutant and/or co-formulation agent and/or carrier.

18. Formulations according to claim 17, wherein the strain is Streptomyces venezuelae DSM 33887.

19. Formulations according to claim 17, wherein the co-formulation agent is Bio-Friendly 1.

20. Formulations according to claim 17, wherein the Streptomyces venezuelae strain is present in the composition in a weight ratio to the total weight (w/w) of the composition from 5% to 75%.

21. Formulations according to claim 20, wherein the Streptomyces venezuelae strain is present in the composition in an amount from 10⁷ to 10⁹ UFC per 1 g of composition.

22. A Streptomyces venezuelae strain referred to as DLS1568, deposited at Leibeniz-Institut DSMZ-Deutsche Sammlung von Mikroorganismen und Zellkulturen GmbH with accession number DSM 33887.

23-24. (canceled)

25. The formulations according to claim 20, wherein the Streptomyces venezuelae strain, is present in the composition in a weight ratio to the total weight (w/w) of the composition from 20% to 40%.

26. The formulations according to claim 20, wherein the Streptomyces venezuelae strain, is present in the composition in a weight ratio to the total weight (w/w) of the composition equal to 30%.

27. Formulations according to claim 21, wherein the Streptomyces venezuelae strain is present in the composition in an amount equal to 10⁸, UFC per 1 g of composition.

28. The method according to claim 9. wherein the strain is Streptomyces venezuelae DSM 33887.

29. The method according to claim 10. wherein the strain is Streptomyces venezuelae DSM 33887.


SEQUENCE LISTING (2 sequences)

SEQ ID NO: 1 - DNA, 19 bp, synthetic construct
  source 1..19: mol_type = other DNA
acaagccctggaaacgggt

SEQ ID NO: 2 - DNA, 19 bp, synthetic construct
  source 1..19: mol_type = other DNA
acgtgtgcagcccaagaca